(19) United States
(12) Patent Application Publication
(10) Pub. No.: US 2016/0175476 A1
(43) Pub. Date: Jun. 23, 2016

(54) IONIC WIND PURIFIER AND HIGH-VOLTAGE PROTECTION DEVICE THEREOF

(71) Applicant: SUZHOU BEIANG TECHNOLOGY LTD., Suzhou (CN)

(72) Inventors: Hongyu Ran, Suzhou (CN); Zhaofeng Wei, Suzhou (CN); Yan Zhang, Suzhou (CN)

(21) Appl. No.: 14/902,824

(22) PCT Filed: Jul. 1, 2014
(86) PCT No.: PCT/CN2014/081340

§ 371 Date: Jan. 4, 2016

(30) Foreign Application Priority Data
Jul. 5, 2013 (CN) 201320400122.0

(51) Int. Cl. A61L 9/22; G05B 9/02; B01D 53/32

(52) U.S. Cl. CPC A61L 9/22; B01D 53/32; G05B 9/02

(57) ABSTRACT

A high-voltage protection device for an ionic wind purifier is described, which includes a micro switch (1) and a contact point (2) matching with the micro switch (1). The micro switch (1) and the contact point (2) are connected in series on a main circuit. Either the micro switch (1) or the contact point (2) is located on an inner side of a door cover (2) of the ionic wind purifier and the other one is located on a contact part of a main body and the door cover (3). The high-voltage protection device can automatically cut off a power source when a door of the ionic wind purifier is opened, so as to ensure the safety of a user. An ionic wind purifier including the high-voltage protection device is also described.

### CROSS-REFERENCE TO RELATED APPLICATIONS

The disclosure claims the priority of Chinese patent application 201320400122.0 filed on Jul. 5, 2013, the disclosure of which is incorporated by reference herein in its entirety.

### TECHNICAL FIELD

The disclosure relates to the field of indoor purification devices, particularly to a high-voltage protection device for an ionic wind purifier. The disclosure further relates to an ionic wind purifier including the high-voltage protection device.

### BACKGROUND

With the improvement of people's living standards, demands for environment protection and health are also rising. It is urgent to develop an air purifier with high performance so as to purify indoor air and optimize an indoor environment, and ionic wind purifiers are also applied more and more widely in everyday life.

A typical ionic wind purifier includes an air inlet, an air outlet, and a generating electrode and a collecting electrode arranged between the air inlet and the air outlet. The generating electrode and the collecting electrode are arranged oppositely. Air passes through a primary filter screen of the air inlet and reaches the generating electrode. The generating electrode forms a plasma field under the action of a high voltage and ionizes some air molecules. When passing through the plasma field, harmful substances including bacteria and the like are oxidized and killed by free radicals having high energy. In the meanwhile, macromolecule organic substances including formaldehyde and the like are also decomposed into water and carbon dioxide by the free radicals having high energy. The partly-ionized air is accelerated by an electric field force to collide with other particles and electrically charge the particles. Most dust and bacteria in a box of the purifier are electrically charged through such a series of snowballing avalanche effect. Electrically charged dust particles moves towards the collecting electrode under the action of the electric field force, and are finally attached to the collecting electrode with an opposite electric charge. Clean air keeps its kinetic energy to continue moving after the electric charges are neutralized, thereby forming an ionic wind so as to circulate the air of a whole space.

It may be learned from the working principle above that a large amount of dust is attached on the collecting electrode and it is necessary to clean a collecting module regularly. Therefore, it is necessary to open a door of the ionic wind purifier frequently so as to take out the collecting module, while the ionic wind purifier therein is provided with a high-voltage component which may be touched by a user accidentally to result in an electric shock if the user has an incorrect operation, i.e., if the user opens the door arbitrarily to take out an electrode without cutting off a power source. Although a high voltage in the purifier is unlikely to cause any serious consequence to the safety of a client, designers should also try to avoid such security risk.

Therefore, it is a problem to be solved by those skilled in the art to add an anti-electric shock protection function to the ionic wind purifier, so as to avoid an electric shock of a user in an incorrect operation and comprehensively ensure the safety of the user.

### SUMMARY

A purpose of the disclosure is to provide a high-voltage protection device for an ionic wind purifier, which can automatically cut off a power source when a door of the ionic wind purifier is opened, thereby avoiding an electric shock on a user and ensuring the safety of the user. Another purpose of the disclosure is to provide an ionic wind purifier including the high-voltage protection device.

The disclosure provides a high-voltage protection device for an ionic wind purifier so as to solve the technical problem above, which includes a micro switch and a contact point matching with the micro switch; wherein the micro switch and the contact point are connected in series on a main circuit of the ionic wind purifier; the micro switch and the contact point are in contact when a door cover of the ionic wind purifier is closed, and the micro switch and the contact point are disconnected when the door cover is opened.

Preferably, either the micro switch or the contact point may be located on an inner side of the door cover of the ionic wind purifier, and other one may be located on a contact part of a main body of the ionic wind purifier and the door cover.

Preferably, one side of the door cover may be rotationally installed on the main body through a rotating shaft, and other side may be locked or unlocked with the main body through a locking component.

Preferably, either the micro switch or the contact point is located on a periphery of the rotating shaft, and other one may be arranged on an intersection of the main body and the rotating shaft.

Preferably, the locking component may include a button exposed on the door cover, a door lock installed on the door cover and linked with the button, and a switch slot matching with the door lock; the switch slot may be opened on the main body.

Preferably, an inner side of the door cover may be provided with an elastic ejector rod contact point and an elastic ejector rod arranged on the main body; the elastic ejector rod may be in a compressed state when the door cover is closed.

Preferably, the high-voltage protection device may further include a grounded metal filter; and the grounded metal filter may be arranged at an air inlet and an air outlet of the ionic wind purifier.

Preferably, a conduction ratio of distance to potential difference between the grounded metal filter and an electrically charged component or an electrode of the ionic wind purifier through a space may be greater than 1 cm/KV.

Preferably, a conduction ratio of distance to potential difference between the grounded metal filter and an electrically charged component or an electrode of the ionic wind purifier through a surface may be greater than 2 cm/KV.

The disclosure further provides an ionic wind purifier, which includes a main body, a door cover which can be opened and closed on the main body, and a corresponding high-voltage protection device. The high-voltage protection device is the high-voltage protection device as described above.

A high-voltage protection device according to the disclosure is applied to an ionic wind purifier. The high-voltage protection device includes a micro switch and a contact point matching with the micro switch, wherein the micro switch and the contact point are connected in series on a main circuit. When the micro switch and the contact point are in contact, the main circuit is closed and an electrically charged component of the ionic wind purifier is energized. When the micro switch and the contact point are separated, the main circuit is disconnected and the electrically charged component is powered off. Either the micro switch or the contact point is located on an inner side of a door cover of the ionic wind purifier, and the other one is located on a contact part of a main body and the door cover, so that the micro switch and the contact point are in contact when the door cover is closed, and are separated when the door cover is opened.

When the door cover is closed, a door lock switch is locked into a door lock switch slot automatically, the contact point will be pressed on the micro switch, the interior of the micro switch is closed and energized, the main circuit of the ionic wind purifier is closed and energized, and the machine may work normally. When the door cover is opened, the micro switch and the contact point are separated, the interior of the micro switch is powered off, the main circuit is disconnected, and the ionic wind purifier is powered off and stops working. In this way, the high-voltage protection device according to the disclosure can cut off a power source automatically when a door of the ionic wind purifier is opened, thereby avoiding an electric shock on a user and ensuring the safety of the user.

In a preferred embodiment, the inner side of the door cover according to the disclosure is provided with an elastic ejector rod contact point and an elastic ejector rod arranged on the main body. The elastic ejector rod is in a compressed state when the door cover is closed. In this way, the elastic ejector rod releases an elastic force after the door cover is unlocked and opens the door cover by ejection so that the door cover is opened automatically, thus the micro switch and the contact point are separated rapidly to turn off the power and the reaction speed of the device is improved.

In another specific embodiment, the high-voltage protection device according to the disclosure further includes a grounded metal filter. The metal filter is arranged at an air inlet/outlet of the ionic wind purifier. In this way, the grounded metal filter is arranged to avoid secondary pollution formed by electrically charged particles which escape out of the purifier and are attached on other surfaces. In the meanwhile, it is ensured that a client will not touch a high-voltage component inside when touching the surface of the purifier, and nor will any static electricity be accumulated on the surface, thereby ensuring absolutely safe use for the client.

### BRIEF DESCRIPTION OF THE DRAWINGS

FIG. 1 is a structural diagram of a high-voltage protection device having a door cover in an opened state according to the disclosure; and

FIG. 2 is a diagram of a setting position of a metal filter according to the disclosure.

### DETAILED DESCRIPTION

The core of the disclosure is to provide a high-voltage protection device for an ionic wind purifier, which can automatically cut off a power source when a door of the ionic wind purifier is opened, thereby avoiding an electric shock on a user and ensuring the safety of the user. Another core of the disclosure is to provide an ionic wind purifier including the high-voltage protection device.

The disclosure will be further expounded hereinafter in conjunction with the accompanying drawings and specific embodiments so that those skilled in the art may better understand the technical solution of the disclosure.

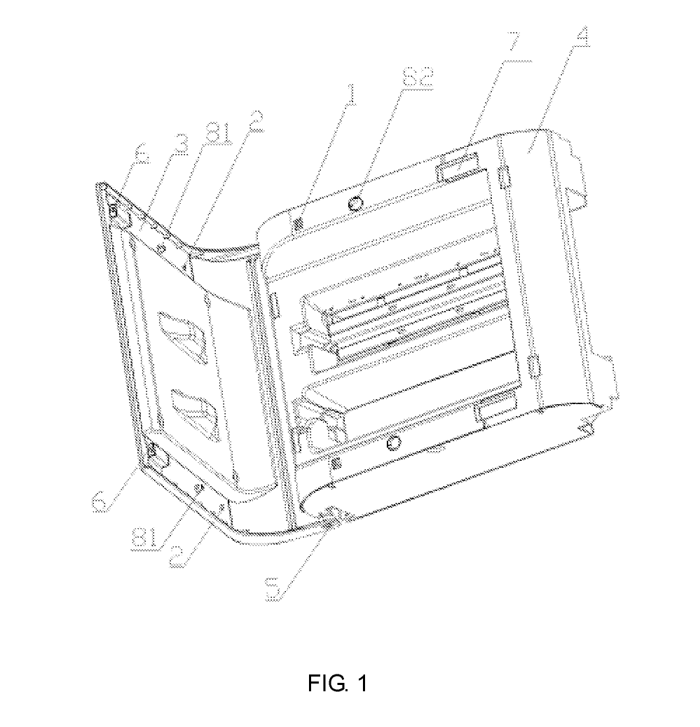

Referring to FIG. 1, FIG. 1 is a structural diagram of a high-voltage protection device having a door cover in an opened state according to the disclosure.

The high-voltage protection device according to the disclosure is applied to an ionic wind purifier. The high-voltage protection device includes a micro switch 1 and a contact point 2 matching with the micro switch 1, wherein the micro switch 1 and the contact point 2 are connected in series on a main circuit. When the micro switch 1 and the contact point 2 are in contact, the main circuit is closed and an electrically charged component of the ionic wind purifier is energized. When the micro switch 1 and the contact point 2 are separated, the main circuit is disconnected and the electrically charged component is powered off. One of the micro switch 1 and the contact point 2 is located on an inner side of a door cover 3 of the ionic wind purifier, and the other one is located on a contact part of a main body 4 and the door cover 3, so that the micro switch 1 and the contact point 2 are in contact when the door cover 3 is closed, and the micro switch 1 and the contact point 2 are separated when the door cover 3 is opened.

The micro switch 1 may be various micro switches conventionally used in the art, such as a button type micro switch or a reed type micro switch and so on.

Preferably, the micro switch 1 is arranged on the contact part and the contact point 2 is arranged on the inner side of the door cover 3 so as to facilitate the arrangement of each component. Apparently, the micro switch 1 may be also arranged on the inner side of the door cover 3 while the contact point 2 is arranged on the contact part, as long as the micro switch 1 and the contact point 2 may be in contact and separated.

When the door cover 3 is closed, a door lock switch is locked into a door lock switch slot 7 automatically, the contact point 2 will be pressed on the micro switch 1, the interior of the micro switch 1 is closed and energized, the main circuit of the ionic wind purifier is closed and energized, and the machine may work normally. When the door cover 3 is opened, the micro switch 1 and the contact point 2 are separated, the interior of the micro switch 1 is powered off, the main circuit is disconnected, and the ionic wind purifier is powered off and stops working. In this way, the high-voltage protection device according to the disclosure can cut off a power source automatically when a door of the ionic wind purifier is opened, thereby avoiding an electric shock on a user and ensuring the safety of the user.

One side of the door cover 3 is rotationally installed on the main body 4 through a rotating shaft 5 and the other side is locked or unlocked with the main body 4 through a locking component. The door cover 3 may rotate around the rotating shaft 5 and may be opened when the door cover 3 and the main body 4 are unlocked.

At the moment, one of the micro switch 1 and the contact point 2 is located on the periphery of the rotating shaft 5 and the other one is arranged on an intersection of the main body 4 and the rotating shaft 5, and they are on initial positions. In other words, the micro switch 1 and the contact point 2 are in contact when the door cover 3 is closed, the main circuit of the device is connected and the device operates normally; when the door cover 3 is opened, the rotating shaft rotates to a certain angle, the micro switch 1 and the contact point 2 are not in contact, the main circuit is disconnected at the moment, and the device is powered off.

The locking component includes a button 6 exposed on the door cover 3, a door lock installed on the door cover 3 and linked with the button 6, and a switch slot 7 matching with the door lock. The switch slot 7 is opened on the main body 4. When the door cover 3 is closed, the door lock is clamped into the switch slot 7 automatically so as to lock the door cover 3. When the button 6 is pressed, the door lock linked with the button 6 is driven to separate from the switch slot 7, thereby unlocking the door cover 3.

Theoretically, the door cover 3 and the main body 4 are not connected only by using the foregoing method, and any connection method is applicable as long as the door cover 3 and the main body 4 can be opened and closed. For example, the door cover 3 and the main body 4 are clamped, and so on.

An inner side of the door cover 3 is provided with an elastic ejector rod contact point 81 and an elastic ejector rod 82 arranged on the main body. The elastic ejector rod 82 is in a compressed state when the door cover 3 is closed. In this way, the elastic ejector rod 82 releases an elastic force when the door cover 3 is unlocked to open the door cover 3 by ejection, so that the door cover 3 is opened automatically, thereby separating the micro switch 1 and the contact point 2 rapidly to turn off the power, and improving the reaction speed of the device.

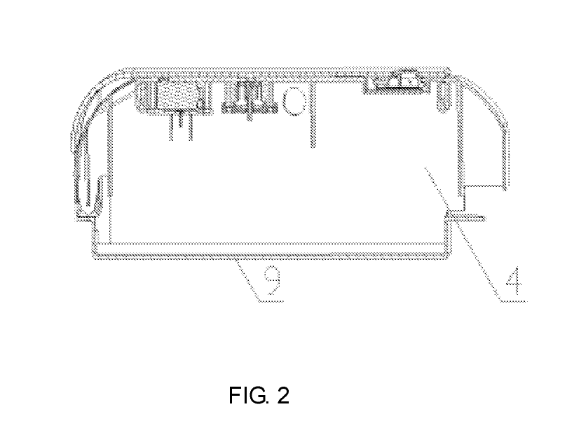

As shown in FIG. 2, the high-voltage protection device according to the disclosure further includes a grounded metal filter 9, and the grounded metal filter 9 is arranged at an air inlet and an air outlet of the ionic wind purifier. In this way, the grounded metal filter 9 is arranged to avoid secondary pollution formed by electrically charged particles which escape out of the purifier and are attached on other surfaces. In the meanwhile, it is ensured that a client will not touch a high-voltage component inside when touching the surface of the purifier, and nor will any static electricity be accumulated on the surface, thereby ensuring absolutely safe use for the client.

A conduction ratio of distance to potential difference between the metal filter 9 and the electrically charged component or an electrode of the ionic wind purifier through a space is greater than 1 cm/KV, and a conduction ratio of distance to potential difference between the grounded metal filter and the electrically charged component or the electrode of the ionic wind purifier through a surface is greater than 2 cm/KV, thereby ensuring safe use of the ionic wind purifier.

Besides the high-voltage protection device, the disclosure further provides an ionic wind purifier including the high-voltage protection device. Referring to the prior art for the structures of other parts of the ionic wind purifier, and description will not be provided repeatedly here.

An ionic wind purifier and a high-voltage protection device thereof according to the disclosure have been expounded above. The principles and embodiments of the disclosure are elaborated herein by applying specific cases. The description of the embodiments is only used for helping a method of the disclosure and the core idea thereof understood. It should be pointed out that several improvements and modifications may be also made to the disclosure without departing from the principles of the disclosure for those of ordinary skill in the art, and these improvements and modifications also fall within the protection scope of the claims of the disclosure.

1. A high-voltage protection device for an ionic wind purifier, comprising a micro switch and a contact point matching with the micro switch; wherein the micro switch and the contact point are connected in series on a main circuit of the ionic wind purifier; the micro switch and the contact point are in contact when a door cover of the ionic wind purifier is closed, and the micro switch and the contact point are disconnected when the door cover is opened.

2. The high-voltage protection device according to claim 1, wherein either the micro switch or the contact point is located on an inner side of the door cover of the ionic wind purifier, and other one is located on a contact part of a main body of the ionic wind purifier and the door cover.

3. The high-voltage protection device according to claim 1, wherein one side of the door cover is rotationally installed on the main body through a rotating shaft, and other side is locked or unlocked with the main body through a locking component.

4. The high-voltage protection device according to claim 3, wherein either the micro switch or the contact point is located on a periphery of the rotating shaft, and other one is arranged on an intersection of the main body and the rotating shaft.

5. The high-voltage protection device according to claim 3, wherein the locking component includes a button exposed on the door cover, a door lock installed on the door cover and linked with the button, and a switch slot matching with the door lock; the switch slot is opened on the main body.

6. The high-voltage protection device according to claim 5, wherein an inner side of the door cover is provided with an elastic ejector rod contact point and an elastic ejector rod arranged on the main body; the elastic ejector rod is in a compressed state when the door cover is closed.

7. The high-voltage protection device according to claim 1, further comprising a grounded metal filter; wherein the grounded metal filter is arranged at an air inlet and an air outlet of the ionic wind purifier.

8. The high-voltage protection device according to claim 7, wherein a conduction ratio of distance to potential difference between the grounded metal filter and an electrically charged component or an electrode of the ionic wind purifier through a space is greater than 1 cm/KV.

9. The high-voltage protection device according to claim 7, wherein a conduction ratio of distance to potential difference between the grounded metal filter and an electrically charged component or an electrode of the ionic wind purifier through a surface is greater than 2 cm/KV.

10. An ionic wind purifier, comprising a main body, a door cover which can be opened and closed on the main body, and a corresponding high-voltage protection device, wherein the high-voltage protection device comprises a micro switch and a contact point matching with the micro switch; wherein the micro switch and the contact point are connected in series on a main circuit of the ionic wind purifier; the micro switch and the contact point are in contact when a door cover of the ionic wind purifier is closed, and the micro switch and the contact point are disconnected when the door cover is opened.

11. The ionic wind purifier according to claim 10, wherein either the micro switch or the contact point is located on an inner side of the door cover of the ionic wind purifier, and other one is located on a contact part of a main body of the ionic wind purifier and the door cover.

12. The ionic wind purifier according to claim 10, wherein one side of the door cover is rotationally installed on the main body through a rotating shaft, and other side is locked or unlocked with the main body through a locking component.

13. The ionic wind purifier according to claim 12, wherein either the micro switch or the contact point is located on a periphery of the rotating shaft, and other one is arranged on an intersection of the main body and the rotating shaft.

14. The ionic wind purifier according to claim 12, wherein the locking component includes a button exposed on the door cover, a door lock installed on the door cover and linked with the button, and a switch slot matching with the door lock; the switch slot is opened on the main body.

15. The ionic wind purifier according to claim 14, wherein an inner side of the door cover is provided with an elastic ejector rod contact point and an elastic ejector rod arranged on the main body; the elastic ejector rod is in a compressed state when the door cover is closed.

16. The ionic wind purifier according to 10, further comprising a grounded metal filter; wherein the grounded metal filter is arranged at an air inlet and an air outlet of the ionic wind purifier.

17. The ionic wind purifier according to claim 16, wherein a conduction ratio of distance to potential difference between the grounded metal filter and an electrically charged component or an electrode of the ionic wind purifier through a space is greater than 1 cm/KV.

18. The ionic wind purifier according to claim 16, wherein a conduction ratio of distance to potential difference between the grounded metal filter and an electrically charged component or an electrode of the ionic wind purifier through a surface is greater than 2 cm/KV.

19. The high-voltage protection device according to claim 3, further comprising a grounded metal filter; wherein the grounded metal filter is arranged at an air inlet and an air outlet of the ionic wind purifier.

20. The ionic wind purifier according to 12, further comprising a grounded metal filter; wherein the grounded metal filter is arranged at an air inlet and an air outlet of the ionic wind purifier.
